(19) United States
(12) Patent Application Publication
(10) Pub. No.: US 2014/0088420 A1
(43) Pub. Date: Mar. 27, 2014

(54) Transducer Storage Rack

(71) Applicant: Glen Thomas Lindahl, Churubusco, IN (US)

(72) Inventor: Glen Thomas Lindahl, Churubusco, IN (US)

(21) Appl. No.: 13/628,524

(22) Filed: Sep. 27, 2012

(51) Int. Cl. A61B 8/00

(52) U.S. Cl. CPC A61B 8/4422
USPC 600/437

(57) ABSTRACT

This disclosure is for a Transducer Storage Rack (FIG. 1) that provides protection for the Transducer Probes(4) and Transducer Connector(3) while not in use. The primary, although not exclusive, use is for Medical Ultrasound Transducer storage. The disclosed design, incorporates two design components in one unit (FIG. 4). The first being a Rack Plate(1), that is mounted to the wall and configured to contain one or more Ultrasound Transducers. The second component is one or more Rack Receptacles(2) that use a locking feature similar to the locking device that retains the signal interface between the Transducer Connector(3) and the Ultrasound System while the Ultrasound Transducer is in use.

### BACKGROUND

The proliferation of Medical Ultrasound systems and the adaptation of specialized Ultrasound Transducers (FIG. 2) have generated the need to safely store Ultrasound Transducers and protect their probes and connectors from damage.

The Ultrasound Transducer incurs damage when the membrane on the face of the Transducer Probe(4) becomes scuffed, nicked, or comes in contact with a hard surface while under external forces. The electrical connections within the Transducer Connector(3) also can be damaged by external force or can develop reduced connectivity due to foreign material contamination while exposed to the atmosphere in the work environment. The potential for damage is increased as the number of contacts within the Transducer Connector(3) increase.

The typical solution, common to the art, is to provide a shelf or cabinet that only allows the unused transducer to be placed out of the way. These previous solutions provide little protection for the Transducer Probe(4), Transducer Cable(5), or the Transducer Connector(3). The design of a Transducer Storage Rack (FIG. 4) disclosed herein provides the protection not provided for in previous designs.

### DESCRIPTION

This disclosure is for a Transducer Storage Rack (FIG. 1) that provides protection for the Transducer Probes(4) and Transducer Connector(3) while not in use. The primary, although not exclusive, use is for Medical Ultrasound Transducer storage.

An Ultrasound Transducer (FIG. 2), common to the art, is comprised of three general components; the Transducer Connector(3) which provides an electrical interface between the Ultrasound System and the Ultrasound Transducer, Transducer Probe(4) which emits and receives ultrasonic signals, and the flexible Transducer Cable(5) which connects the Transducer Probe(4) to the Transducer Connector(3).

The disclosed design, in its basic implementation incorporates two design components in one unit (FIG. 4). The first being a Rack Plate(1), that is mounted to the wall and configured to contain one or more Ultrasound Transducers. The second component is one or more Rack Receptacles(2) that use a locking feature similar to the locking device that retains the signal interface between the Transducer Connector(3) and the Ultrasound System while the Ultrasound Transducer is in use. A simple locking feature could be composed of a rotating cross pin that fits into a slotted hole to secure the Transducer Connector (3) to an Ultrasound System or, in this disclosure, a Transducer Storage Rack (FIG. 3). These components together provide protection and security for the Transducer Probes(4) and the Transducer Connectors(3) (FIG. 1).

In the preferred implementation of this design (FIG. 4) the Rack Receptacles(2) are discrete components, allowing for different Rack Receptacle(2) designs to be implemented within one Transducer Storage Rack. That design consideration does not preclude the incorporation of Rack Receptacle(2) features into the, Rack Plate(1) if alternate manufacturing processes were used to produce the Transducer Storage Rack, and multiple receptacle designs were not required within a single Transducer Storage Rack.

In the preferred implementation of this disclosure these Rack Receptacles(2)are located below, and offset (FIG. 5), from the Transducer Probes(4), providing less stress on the Electrical Cable(5).

### DRAWING DESCRIPTION

FIG. 1

Disclosed Transducer Storage Rack(1) depicted in use. With a Transducer Connector(3) locked onto a Rack Receptacle(2) (See also FIGS. 3 & 4), and the Transducer Probe(4) stored above and offset from the Transducer Connector(3). The Transducer Cable(5) is allowed to drape naturally between the Transducer Connector(3) and the Transducer Probe(4).

FIG. 2

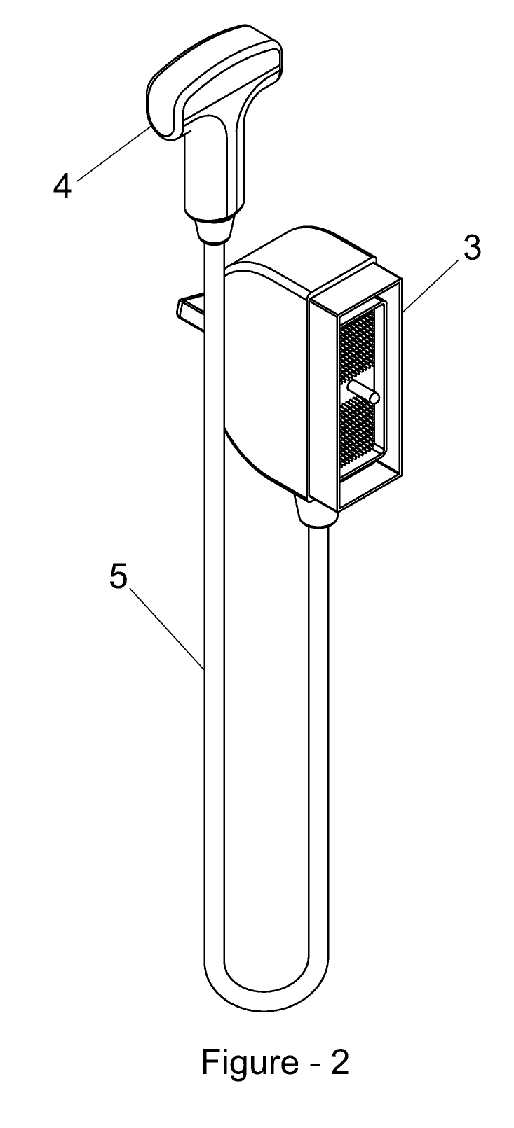

Typical Ultrasound Transducer. Transducer Connector (3), Transducer Probe(4) and Transducer Cable(5) showing the electrical connector side of the Transducer Connector (3) with its multiple contacts and locking pin. In this particular type of Transducer Connector(3) a knob is rotated, rotating a shaft and cross pin that locks the Transducer Connector (3) in place, maintaining electrical contact between the Transducer Connector (3) and the Ultrasound System it is used with.

FIG. 3

Depicted is the Transducer Connector (3) and the Rack Receptacle(2) showing the conformity of the Rack Receptacle(2) to the Transducer Connector (3).

FIG. 4

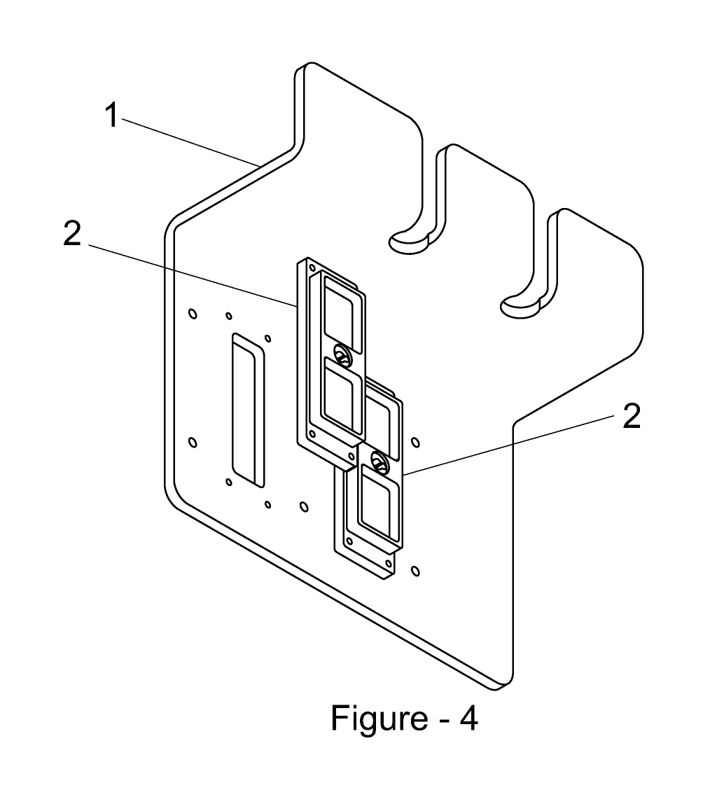

Exploded view of the Rack Plate(1) and the Rack Receptacle(2).

FIG. 5

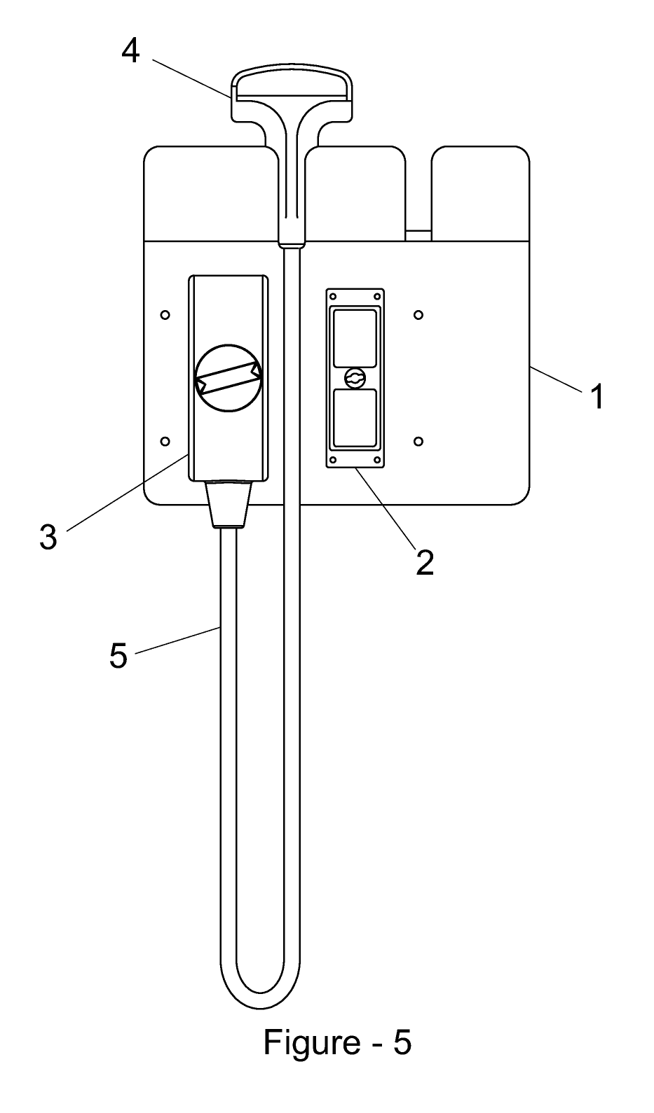

Front orthogonal view of Transducer Storage Rack and Ultrasound Transducer depicting Transducer Probe(4) storage offset from Transducer Connector(3) storage location when locked onto the Rack Receptacle(2).

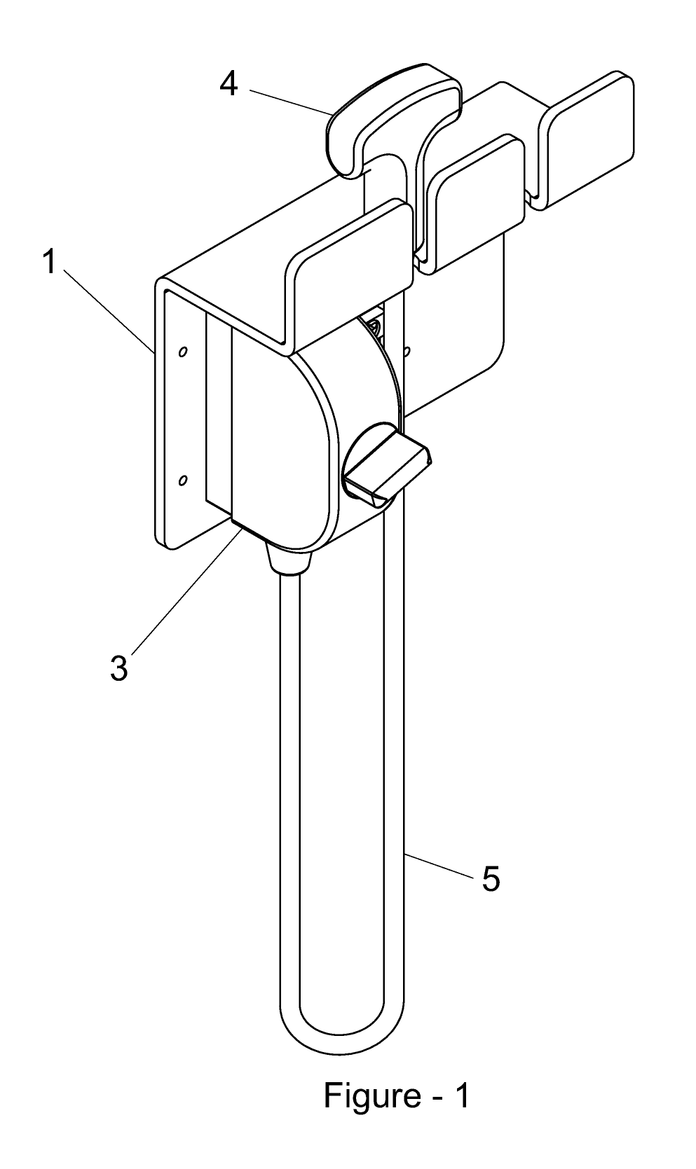

1. This disclosure describes the implementation of a storage device for Medical Ultrasound Transducers in particular (FIG. 1), and transducers in general, that protects the electrical connections within the Transducer Connector(3) from contamination and damage, while providing a safe storage capability for the Transducer Probe(4) and reduced stress on the Electrical Cable(5) between the Transducer Probe(4) and the Transducer Connector(5).

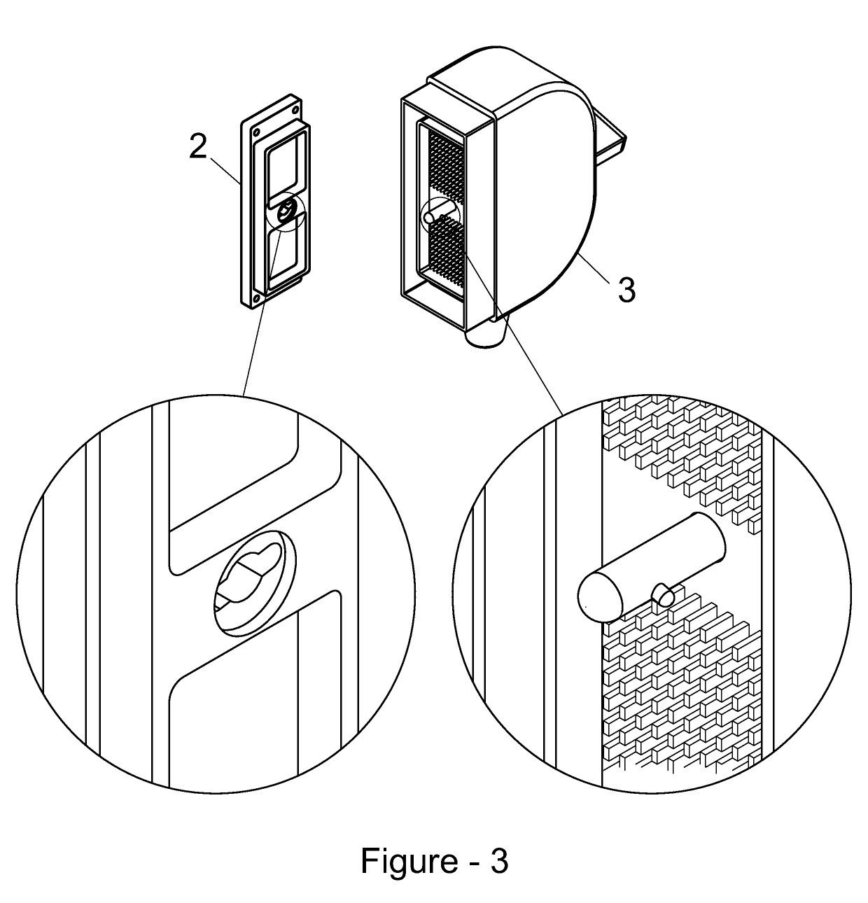

2. Also claimed is that this design includes a Rack Receptacle(2) that incorporates a locking facility (FIG. 3) that retains the Transducer Connector(3) onto the Rack Receptacle(2). This locking feature is similar in function to the locking apparatus used to secure the Transducer Connector(3) to the Ultrasound System when in use.

3. The locking facility incorporated to secure the Transducer Connector(3) onto the Rack Receptacle(2), in this disclosure, can be either passive or active.

4. The design disclosed herein could be implemented as a one piece molding providing the facility that would include the Rack Plate(1) and one or more Rack Receptacles(2).

5. The implementation of this Transducer Storage Rack incorporates a Probe storage feature laterally offset, either above or below the Rack Receptical(2), providing reduced stress and damage to the connecting Electrical Cable(5) while Ultrasound Transducer is in storage.
